(19) United States
(12) Patent Application Publication
(10) Pub. No.: US 2026/0183150 A1
(43) Pub. Date: Jul. 2, 2026

(54) Off-Loading Device for Sacral Ulcers

(71) Applicant: David Elliot Shaffer, Great Neck, NY (US)

(72) Inventor: David Elliot Shaffer, Great Neck, NY (US)

(21) Appl. No.: 19/127,366

(22) PCT Filed: Nov. 2, 2023
(86) PCT No.: PCT/US2023/036718

§ 371 Date: May 5, 2025

Related U.S. Application Data
(60) Provisional application No. 63/382,485, filed on Nov. 4, 2022.

(51) Int. Cl. A61F 13/05; A61F 13/00; A61F 13/0246; A61F 13/06; A61F 13/10; A61F 13/12

(52) U.S. Cl. CPC A61F 13/05; A61F 13/00063; A61F 13/0253; A61F 13/069; A61F 13/101; A61F 13/12

(57) ABSTRACT

An off-loading device for sacral ulcers includes a pressure relieving pad, an adhesive layer, and a protective film. The pressure relieving pad includes a flat pad body and at least one perforated line. The adhesive layer is superimposed onto a distal face of the flat pad body so that the pressure relieving pad can be attached to the patient's skin. The protective film is removably superimposed onto the adhesive layer to protect the adhesive layer. The flat pad body includes a central section and at least one peripheral section, wherein the peripheral section is concentrically positioned around the central section and perimetrically attached to the central section by the perforated line. Once the central section is separated from the peripheral section through the perforated line, the peripheral section can be adhered around the sacral ulcers for treatment or patient's skin can be elevated for prevention of the sacral ulcers.

The current application claims a priority to the U.S. provisional patent application Ser. No. 63/382,425 filed on Nov. 4, 2022.

### FIELD OF THE INVENTION

The present invention relates generally to an apparatus for an off-loading device. More specifically, the present invention is an off-loading device for the prevention and treatment of sacral ulcers.

### BACKGROUND OF THE INVENTION

When it comes to the standard of care in healthcare, there are certain injuries that should never occur, these are called “Never Events”. One injury that health insurance companies deem to be a “Never Events” in nursing homes, hospitals, home health scenarios, is a pressure sore/ulcer. The majority of all pressure sores are preventable and health insurance companies can deny and reduce payments for ulcer treatment if the ulcer develops due to negligence or is not treated in a timely manner. While it might not be possible to prevent all types of pressure related injuries from forming, stage 3 and stage 4 ulcers are considered entirely preventable. Bedsores can develop rather quickly and must be immediately addressed. If left ignored and untreated, the sore may progress to cellulitis, sepsis, infection of bones and joints, and even result into a cancer called Squamous Cell Carcinoma. Pressure related injuries, including sacral ulcers, result from pressure applied to a body part, preventing blood from reaching the skin. In the case of sacral ulcers, a skin injury occurs in the region of the body where the lower back and the spine meet. These pressure sores are also known as bedsores. These bedsores are a preventable event that result in unnecessary pain and suffering, generally developing from patient neglect. The sacral area is subject to substantial friction and shearing forces. These injuries are often a result of extended sitting and/or lying down. The US Centers for Disease Control and Prevention (CDC) reports that 1 in 10 nursing home residents has a pressure related injury to the skin. Some of the contributing factors that are likely to cause sacral ulcers are: immobility, excessive moisture, and poor diet. In the United States, pressure related injuries are scored by stages. (1-4). Nearly half of all the patients are categorized as late stage 3 & 4.

Late-stage pressure sores require longer healing time and continued medical treatment. Pressure sores, as well as sacral ulcers, are treated according to their severity. Stage 1: Skin irritation / discoloration with possible mild tenderness to touch. Stage 2:
Skin begins to die, indent at pressure site, blister, or leakage evident. Stage 3: Open sore extending through several layers of skin to fat (smell, pus, dead tissue). Stage 4: damage to tendons, muscles, & bone.

Areas that are at high risk for developing Pressure related injuries include: sacral, shoulder, knee, heel, elbow, ankles, hip, back of the head, and feet. Spinal cord injuries are at a higher risk of developing pressure sores with complications leading to infections, amputations, loss of work and wages, along with increased medical expenses. The majority of individuals with spinal cord injuries have a pressure related sore. The elderly and bed ridden are prone to pressure sores because they often lie in bed for extended periods of time, resulting in pressure sores forming. People who require the use of a wheelchair or are confined to a bed should make prevention and treatment of pressure sores a priority in their care. Most people who get a bed sore have limited mobility, therefore cannot change positions periodically. (i.e., Spinal Cord Injuries & Coma) Neurological disorders can result in a decrease in sensory perception, thus contributing to PU. Poor nutrition, dehydration, diabetes, and peripheral vascular disease can cause an increase in tissue breakdown.

It is therefore an objective of the present invention to provide an off-loading device for sacral ulcers so that the pressure related injuries can be elevated for treatment or pressure related injuries can be prevented. A patient can have the best available doctors, the appropriate antibiotic therapy, proper wound debridement, but if the bony prominence is not off-loaded and pressure is dispersed, the pressure sore never heals and may result in delayed healing of the wound. Reducing the abnormal pressure over the bony prominence is the key to both prevention and treatment of these soft tissue injuries. Pressure ulcers can be prevented. Not every patient entering the hospital or nursing home may be a candidate for an off-loading device. However, high risk patients should use off-loading devices to reduce the risk of pressure related injuries in patients with one or more of the following: 1.) Diabetics and those that manifest neuropathy; 2.) Diabetics with peripheral vascular disease; 3.) Patients with poor or limited mobility (e.g., hip fracture, stroke, paralysis, unconsciousness, and limited mobility in a bed or wheelchair); 4.) Patients undergoing multi hour surgeries, immobility in the recovery room, and bed for hours afterwards; 5.) Patients with severe PVD with no popliteal pulse; 6.) Patients with a past history of pressure sores might be seen with poor nutrition, spasticity, contractures or agitations; and 7.) Patients with a Braden Score of 15 or less (Braden Score predicts pressure ulcer risk).

### BRIEF DESCRIPTION OF THE DRAWINGS

FIG. 1 is a top perspective view of the present invention.

FIG. 2 is a bottom perspective view of the present invention.

FIG. 3 is a top view of the present invention.

FIG. 4 is a side view of the present invention.

FIG. 5 is a detailed view of the present invention taken about circle 5 in FIG. 4.

FIG. 6 is a schematic view of the present invention showing the first quantity of bioceramics within the flat pad body.

FIG. 7 is a schematic view of the present invention showing the second quantity of bioceramics within the adhesive layer.

FIG. 8 is a top view of the present invention, wherein the at least one peripheral section is the plurality of peripheral sections, and the at least one perforated line is the plurality of perforated lines.

### DETAILED DESCRIPTION OF THE INVENTION

All illustrations of the drawings are for the purpose of describing selected versions of the present invention and are not intended to limit the scope of the present invention.

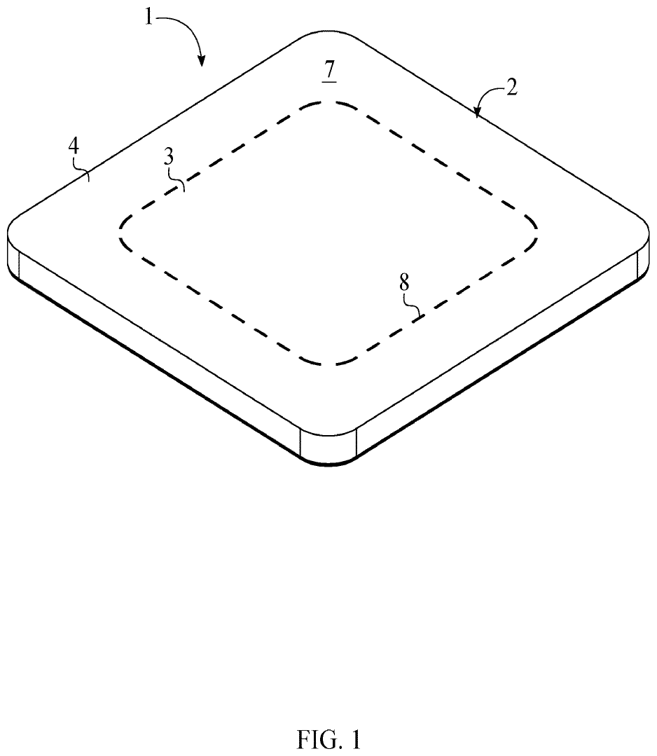

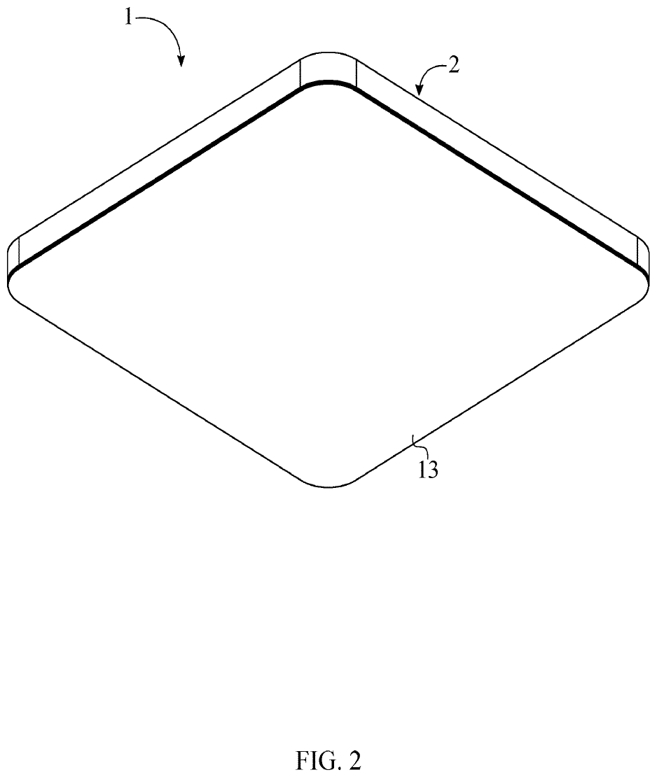

Approximately 60,000 patients die each year as a direct result of pressure sore. That is equivalent to 6.85 deaths every hour, every single day of the year. More than 17,000 lawsuits are related to the pressure sore injuries annually thus resulting in an average settlement of around 400,000 dollars. It is estimated that 2.5 million people per year are affected with pressure related injuries and the cost of treating the pressure sore in the US ranges from 9.1 to 11.6 billion dollars per year. The average individual cost ranges from 20,000-151,000 dollars. Medicare estimates that each of the pressure sore adds 43,000 dollars to the cost of a hospital stay. Statistically, bedsores are more often found in nursing homes as compared to hospitals and homecare. This is probably a direct result of understaffing in the Nursing Home Industry. Not enough nurses translate into placing the patient at risk from being repositioned. The most common cause of skin breakdown is the failure to turn and reposition patients. The second leading cause of the pressure sore is dehydration and malnutrition. As nursing homes and hospitals push to maximize bottom line profits through restricting expenses and reducing work staff, pressure related injuries tend to increase. The present invention reduces pressure, friction, and shearing forces over pressure sensitive areas of the skin thus providing adjustability to relieving pressure over bony prominences and other effected skin area. As shown in FIGS. 1-4, the present invention comprises a pressure relieving pad 1, an adhesive layer 12, and a protective film 13.

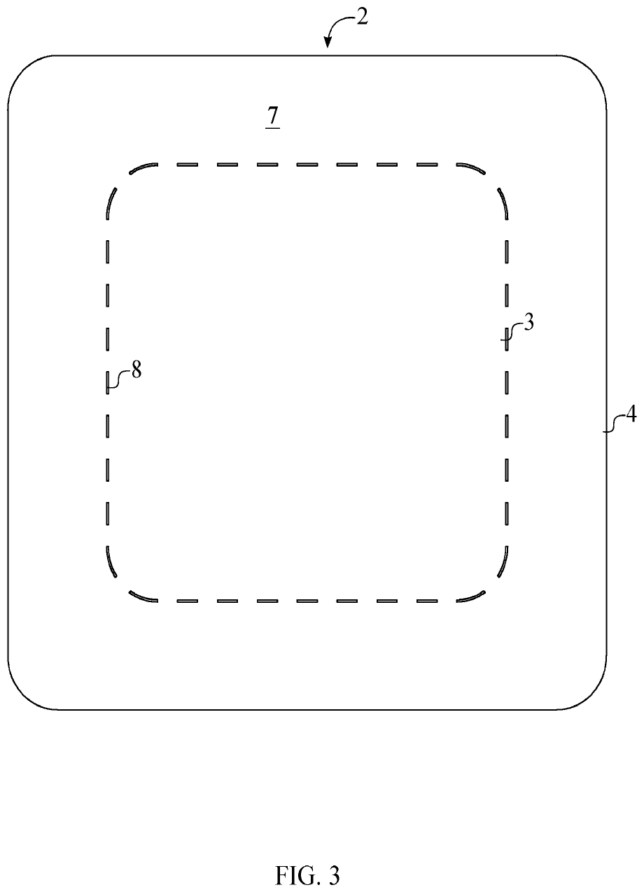

Preferably, the pressure relieving pad 1 is shaped into a rectangular shape as shown in FIG. 3. However, the pressure relieving pad 1 can be formed in any organic shapes or geometric shapes such as circular, triangular, and rectangular. In reference to FIGS. 1-3, the pressure relieving pad 1 comprises a flat pad body 2 and at least one perforated line 8 as the flat pad body 2 is sectionalized via the at least one perforated line 8. The flat pad body 2 is a thickness pad and comprises a central section 3, at least one peripheral section 4, a proximal face 6, and a distal face 7. More specifically, the least one peripheral section 4 is concentrically positioned around the central section 3, and the least one peripheral section 4 is perimetrically attached to the central section 3 by the at least one perforated line 8.

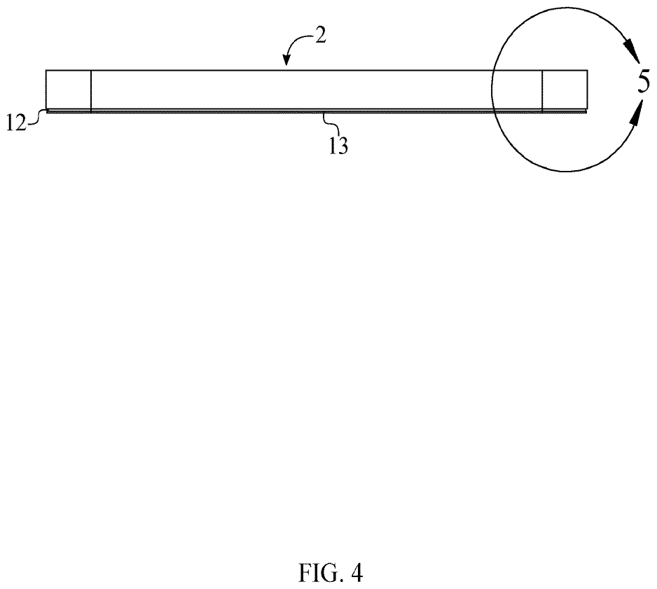

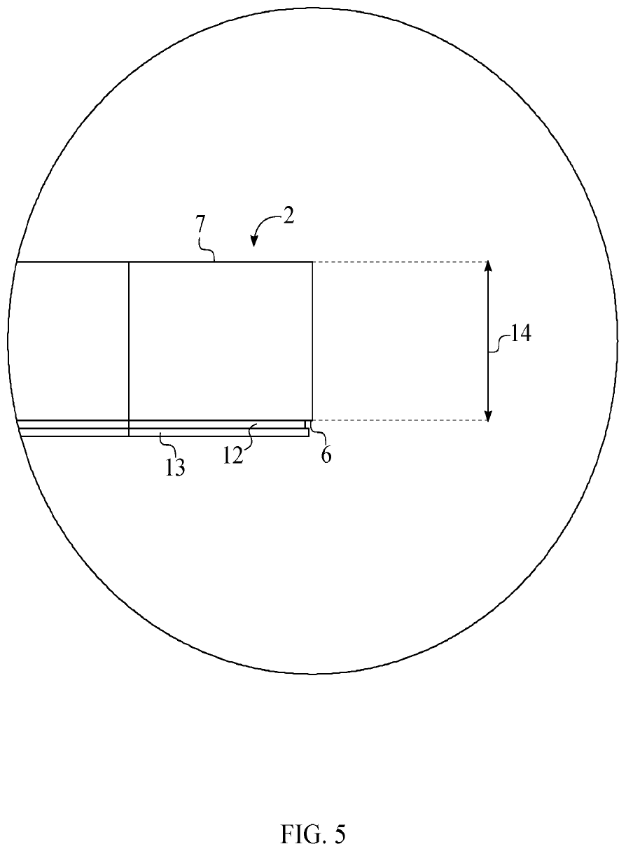

In reference to FIG. 4 and FIG. 5, the thickness of the flat pad body 2 can vary according to how much pressure relieving is necessary and the physical arrangement of the human body. In other words, the distal face 7 is positioned offset to the proximal face 6 as a first distance 14 is delineated between the distal face 7 and the proximal face 6 and defines the thickness of the flat pad body 2. Preferably, the first distance 14 is approximately 1 centimeter so that the flat pad body 2 is able to sufficiently elevate patient's sink and provide optimal off-loading dispersion. However, the first distance 14 is not limited to 1 centimeter and can be less than 1 centimeter or greater than 1 centimeter depending upon the exact physical positioning of the sacral ulcer or the weight of the patient. The flat pad body 2 can be made of foam, rubber, neoprene, Polypropylene Terephthalate (PPT), polyurethane, polystyrene, Polyvinyl Chloride (PVC), or any other materials that can mimic skin and reduce pressure over pressure sensitive areas. Preferably, the flat pad body 2 is made of a medical-grade foam to provide a clean or sterile environment for the patient's skin and the surrounding skin of the ulcer. Furthermore, the medical-grade foam can absorb exudate and fluid directly from an ulcer wound and as well as the surrounding area.

In reference to FIG. 5, the adhesive layer 12 is superimposed onto the proximal face 6 of the flat pad body 2 so that the flat pad body 2 can be adhered to the patient's skin and around the sacral ulcer, over clothing material, dressing, or bandage. Furthermore, the adhesive layer 12 provides painless and comfortable adhering properties to reduce irritations to patient's skin. Preferably, the adhesive layer 12 is made of a medical-grade adhesive to provide a clean or sterile environment for the patient's skin. In order to protect the adhesive properties of the adhesive layer 12 and to maintain a sterile surface, the protective film 13 is removably superimposed onto the adhesive layer 12. Resultantly, the protective film 13 has to be removed before the flat pad body 2 is adhered to the patient's skin.

The flat pad body 2 is utilized in the prevention and treatment of pressure sores to relieve pressure over ulcerative areas, prominent bony areas of the sacral area, shoulder, knee, heels, ankles, feet, elbows, hip, neck, leg, back, head, and any other sensitive skin areas in the human body. Because of the at least one perforated line 8, a caregiver can easily remove the central section 3 from the at least one peripheral section 4 so that the at least one peripheral section 4 can be adhered around the sacral ulcer. Resultantly, non-bulky construction of the at least one peripheral section 4 is able to reduce the pressure over soft tissue, bony prominences, or sacral ulcers.

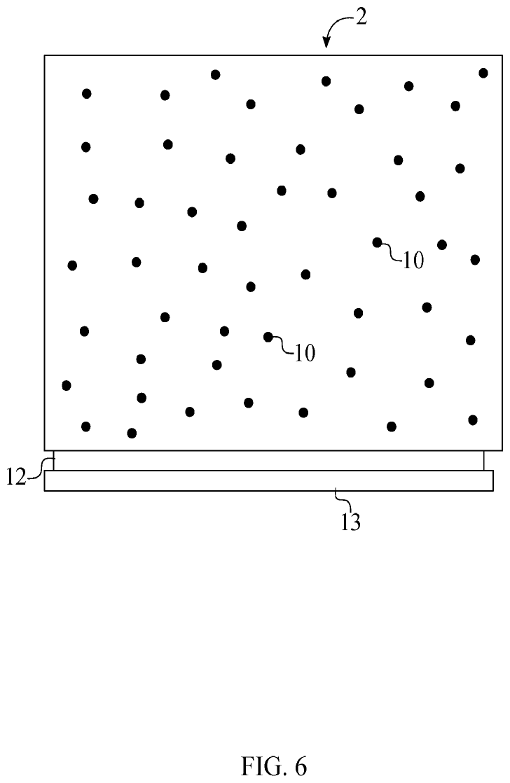

In reference to FIG. 6, the present invention may further comprise a first quantity of bioceramics 10. The first quantity of bioceramics 10 is integrated into the flat pad body 2 during the manufacturing process or after the manufacturing process. More specifically, the first quantity of bioceramics 10 is configured to thermally insulate body heat in and around an ulcer in order to improve blood circulation in and around the ulcer and to consequently improve healing of the ulcer. In other words, the first quantity of bioceramics 10 improves the wound healing properties in addition to the functionality of the flat pad body 2.

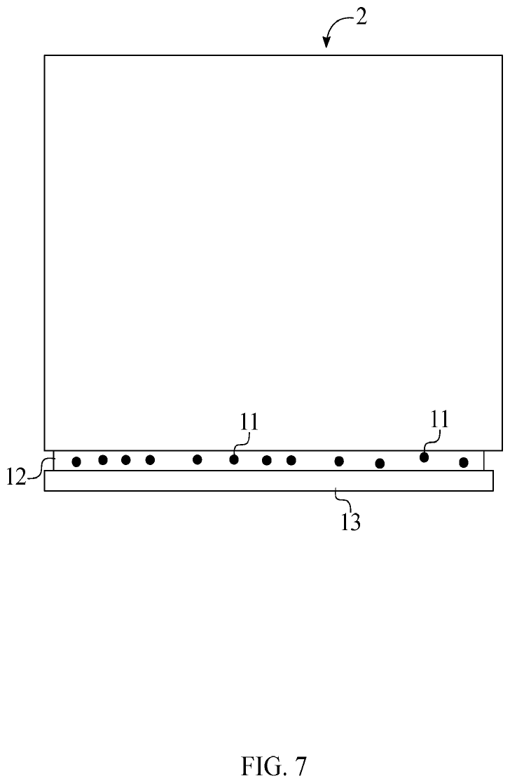

In reference to FIG. 7, the present invention may further comprise a second quantity of bioceramics 11. The second quantity of bioceramics 11 is integrated into the adhesive layer 12 during the manufacturing process or after the manufacturing process. More specifically, the second quantity of bioceramics 11 is configured to thermally insulate body heat in and around an ulcer in order to improve blood circulation in and around the ulcer and to consequently improve healing of the ulcer. In other words, the second quantity of bioceramics 11 improves the wound healing properties in addition to the functionality of the adhesive layer 12.

Even though the central section 3 is supposed to be discarded, the central section 3 can optionally be adhered adjacent to sacral ulcer to improve blood circulation when the first quantity of bioceramics 10 is present within the central section 3 or the second quantity of bioceramics 11 is present within the adhesive layer 12.

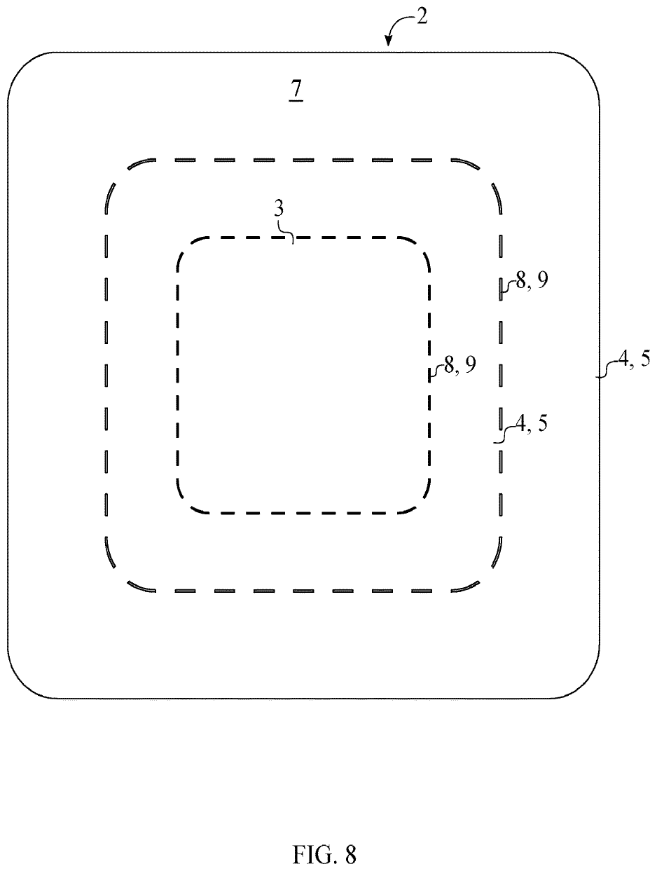

As shown in FIG. 8, the at least one peripheral section 4 can be a plurality of peripheral sections 5, and the at least one perforated line 8 can be a plurality of perforated lines 9 to accommodate to the varying sizes of ulcers presented. More specifically, the plurality of peripheral sections 5 is a plurality of closed-loop concentric sections. The plurality of perforated lines 9 is a plurality of closed-loop concentric lines. The central section 3 and a diametrically-smallest peripheral section from the plurality of peripheral sections 5 are delineated from each other by a diametrically-smallest perforated line from the plurality of perforated lines 9. Each adjacent pair of peripheral sections from the plurality of peripheral sections 5 is delineated from each other by a corresponding intermediate perforated line from the plurality of perforated lines 9. As a result, the caregiver can remove the central section 3 and then remove any number of peripheral sections 5 so that the flat pad body 2 can be completely positioned and adhered around the sacral ulcer.

Resultantly, the present invention is able to prevent, minimize, and/or treat pressure related injuries in hospitals, nursing homes, and homecare services thus extending health and wellbeing of the patients. At the same time, the present invention is able to save considerable amounts of money for the patient that is generally associated with the pressure related injuries. Even though the present invention is specifically explained in relation to sacral ulcers, the present invention is not limited to sacral ulcers and can be utilized to reduce pressure over any sensitive wound areas, sensitive skin areas, and any other type of ulcers.

Although the invention has been explained in relation to its preferred embodiment, it is to be understood that many other possible modifications and variations can be made without departing from the spirit and scope of the invention as hereinafter claimed.

What is claimed is:

1. An off-loading device for sacral ulcers comprising:
a pressure relieving pad;
an adhesive layer;
a protective film;
the pressure relieving pad comprising a flat pad body and at least one perforated line;
the flat pad body comprising a central section, at least one peripheral section, a proximal face, and a distal face;
the adhesive layer being superimposed onto the proximal face of the flat pad body;
the protective film being removably superimposed onto the adhesive layer;
the least one peripheral section being concentrically positioned around the central section; and
the least one peripheral section being perimetrically attached to the central section by the at least one perforated line.

2. The off-loading device for sacral ulcers as claimed in claim 1, wherein the flat pad body is made of a medical-grade foam.

3. The off-loading device for sacral ulcers as claimed in claim 1, wherein the adhesive layer is made of a medical-grade adhesive.

4. The off-loading device for sacral ulcers as claimed in claim 1 comprising:
a first quantity of bioceramics; and
the first quantity of bioceramics being integrated into the flat pad body.

5. The off-loading device for sacral ulcers as claimed in claim 4, wherein the first quantity of bioceramics is configured to thermally insulate body heat in and around an ulcer in order to improve blood circulation in and around the ulcer and to consequently improve healing of the ulcer.

6. The off-loading device for sacral ulcers as claimed in claim 1 comprising:
a second quantity of bioceramics; and
the second quantity of bioceramics being integrated into the adhesive layer.

7. The off-loading device for sacral ulcers as claimed in claim 6, wherein the second quantity of bioceramics is configured to thermally insulate body heat in and around an ulcer in order to improve blood circulation in and around the ulcer and to consequently improve healing of the ulcer.

8. The off-loading device for sacral ulcers as claimed in claim 1 comprising:
the distal face being positioned offset to the proximal face;
a first distance being delineated between the distal face and the proximal face; and
the first distance being approximately 1 centimeter.

9. The off-loading device for sacral ulcers as claimed in claim 1 comprising:
the at least one peripheral section being a plurality of peripheral sections;
the at least one perforated line being a plurality of perforated lines;
the plurality of peripheral sections being a plurality of closed-loop concentric sections;
the plurality of perforated lines being a plurality of closed-loop concentric lines;
the central section and a diametrically-smallest peripheral section from the plurality of peripheral sections being delineated from each other by a diametrically-smallest perforated line from the plurality of perforated lines; and
each adjacent pair of peripheral sections from the plurality of peripheral sections being delineated from each other by a corresponding intermediate perforated line from the plurality of perforated lines.

10. The off-loading device for sacral ulcers as claimed in claim 1, wherein the flat pad body is utilized in the prevention and treatment of pressure sores to relieve pressure over ulcerative areas, prominent bony areas of the sacral area, shoulder, knee, heels, ankles, feet, elbows, hip, neck, leg, back, head, and any other sensitive skin areas in the human body.
